(19) United States
(12) Patent Application Publication
(10) Pub. No.: US 2022/0099538 A1
(43) Pub. Date: Mar. 31, 2022

(54) APPARATUS FOR TAKING A SAMPLE OF MILK THAT FLOWS THROUGH A MILK LINE

(71) Applicant: Nedap N.V., Groenlo (NL)

(72) Inventors: Edwin SCHAPERCLAUS, Neede (NL); Rudie Jan Hendrik LAMMERS, Eibergen (NL); Jort Johannes Wilhelmus SCHUTTE, Heeten (NL)

(21) Appl. No.: 17/425,278

(22) PCT Filed: Jan. 29, 2020
(86) PCT No.: PCT/NL2020/050046

§ 371 Date: Jul. 22, 2021

(30) Foreign Application Priority Data
Jan. 31, 2019 (NL) 2022487

(51) Int. Cl. G01N 1/20; G01N 33/04; A01J 5/04

(52) U.S. Cl. CPC G01N 1/2035; G01N 2001/205; A01J 5/045; G01N 33/04

(57) ABSTRACT

An apparatus (1) for taking a sample (2) of milk (4) that flows through a milk line (6), wherein the apparatus comprises a main valve (8) comprising a first valve inlet (10) and a first valve outlet (12), the apparatus being configured for including the main valve in a milk flow path of the milk line by placing the first valve inlet in fluid communication with a first open end (16) of the milk line and placing the first valve outlet in fluid communication with a second open end (18) of the milk line, the first open end being located upstream of the second open end in the milk flow path, the apparatus further comprising a sample collecting chamber (20) for receiving the sample of the milk, the sample collecting chamber being provided with a sample collecting chamber inlet (22) and at least one sample collecting chamber outlet (24), and the main valve further comprising a second valve outlet (26), the second valve main valve is configured to selectively swatch between a first and a second state, wherein in the first state the first valve inlet is in fluid communication with the first valve outlet so that the milk can flow through the milk line without a sample being taken and wherein in the second state the first valve inlet is in fluid communication with the second valve outlet for supplying milk from the milk line to the sample collecting chamber and wherein the apparatus is configured to selectively place or not place the sample collecting chamber outlet in fluid communication with the milk line downstream of the main valve for leading back to the milk line excess milk of a milk sample which is in the sample collecting chamber.

The invention relates to an apparatus for taking a sample of milk that flows through a milk line. The object of the invention is to provide an apparatus that can be simply included in the milk line so that in an easy manner a sample of the milk that flows through the milk line can be taken.

To this end, the apparatus according to the invention is characterized in that the apparatus comprises a main valve comprising a first valve inlet and a first valve outlet, the apparatus being configured for including the main valve in a milk flow path of the milk line by placing the first valve inlet in fluid communication with a first open end of the milk line and placing the first valve outlet in fluid communication with a second open end of the milk line, the first open end being located upstream of the second open end in the milk flow path, the apparatus further comprising a sample collecting chamber for receiving the sample of the milk, the sample collecting chamber comprising a sample collecting chamber inlet and a sample collecting chamber outlet, and the main valve further comprising a second valve outlet, the second valve outlet being in fluid communication, for example through at least a first line, with the sample collecting chamber inlet, wherein the main valve is configured to selectively switch between a first and second state, wherein in the first state the first valve inlet is in fluid communication with the first valve outlet so that the milk can flow through the milk line without a sample being taken and wherein in the second state the first valve inlet is in fluid communication with the second valve outlet for supplying milk from the milk line to the sample collecting chamber and wherein the apparatus is configured to selectively place or not place the sample collecting chamber in fluid communication, for example through a second line in which a first switchable open-close valve is included, with the milk line downstream of the main valve for leading back to the milk line excess milk of a milk sample which is in the sample collecting chamber.

By virtue of the use of the main valve, as desired, a sample or no sample may be taken without drastic operations with regard to the milk line being necessary. When the valve is in the first state, the milk can flow in the milk line without a sample being taken, whereas, when the valve is in the second state, a sample can be taken from the milk line. Moreover, with the aid of the first switchable open-close valve, excess milk that is in the sample collecting chamber can be led back to the milk line. For installation of the apparatus, the milk line needs to be interrupted (cut through) only at two points at most, and in particular only at one point.

According to a first variant, it holds in particular that the apparatus further comprises a milk flow-through unit provided with a milk flow-through unit inlet, a first milk flow-through unit outlet and a second milk flow-through unit outlet, wherein the second valve outlet is in fluid communication, for example through a third line, with the milk flow-through unit inlet and the first milk flow-through unit outlet is in fluid communication, for example through a fourth line, with the sample collecting chamber inlet so that the fluid communication between the second valve outlet and the sample collecting chamber inlet extends through the milk flow-through unit and wherein the second milk flow-through unit outlet is in fluid communication, for example through a fifth line, with the milk line downstream of the main valve, the milk flow-through unit being so configured that a milk stream which is supplied to the milk flow-through unit inlet of the milk flow-through unit splits up into a first milk stream and a second milk stream which leave the milk flow-through unit respectively via the first milk flow-through unit outlet and the second milk flow-through unit outlet, the flow of the first and second milk stream having a predetermined ratio so that, in use, when the main valve is in the second state, a portion of the milk which flows through the second valve outlet is supplied to the sample collecting chamber by way of the first milk stream and a portion of the milk which flows through the second valve outlet is supplied to the milk line downstream of the main valve by way of the second milk stream for return of this milk to the milk line.

In this manner, when so desired, a sample can be composed from milk which can be obtained spread in time, for example spread over the complete milk yield in one milking of a cow. The flow of the first milk stream is then equal to, for example, 1.5% to 4%, in particular 2%, of the flow of the milk that is supplied to the first milk inlet of the main valve.

A complete milk yield varies from 2-40 liters so that when the first milk stream is 2% of the flow mentioned, the sample has a magnitude of 2/50-40/50 liter.

According to a first embodiment of the last-mentioned variant, it holds that the milk flow-through unit comprises a chamber with a ceiling, a first upstanding line section which comprises an open end at its upper side and of which an open underside is connected with the first milk flow-through unit outlet, a second upstanding line section which comprises an open end at its upper side and of which an open underside is connected with the milk flow-through unit inlet and a bottom having an outflow opening which is connected with the second milk flow-through unit outlet, the milk flow-through unit being so configured that a milk stream which is supplied to the milk flow-through unit inlet is, inside the chamber, upwardly directed and via the second upstanding line section spouts against the ceiling to then leave the milk flow-through unit respectively via the open end at the upper side of the first upstanding line section, the rest of the first upstanding line section and the first milk flow-through unit outlet as the first milk stream and via the outflow opening and the second milk flow-through unit outlet as the second milk stream.

According to an alternative embodiment of the last-mentioned variant, it holds that the milk flow-through unit comprises a chamber provided with the milk flow-through unit inlet, the first milk flow-through unit outlet and the second milk flow-through unit outlet, the milk flow-through unit inlet and the second milk flow-through unit outlet lying opposite each other and the first milk flow-through unit outlet lying between the milk flow-through unit inlet and the second milk flow-through unit outlet, the milk flow-through unit being so configured that a milk stream which is supplied to the milk flow-through unit inlet leaves the milk flow-through unit via the first milk flow-through unit outlet in the first milk stream and via the second milk flow-through unit outlet in the second milk stream.

According to the invention, the sample can also be taken in a different manner than discussed in the foregoing.

According to this variant, it holds that the second valve outlet is directly, for example via the first line, in fluid communication with the sample collecting chamber inlet, the apparatus further comprising a control unit for controlling the main valve from its first state to its second state and vice versa. Here, it holds in particular that the control unit controls the main valve from its first state to its second state and vice versa with a pattern such that the sample collecting chamber, in use, is filled with partial samples which in combination represent a picture of the total amount of milk that flows through the valve inlet of the main valve.

According to yet another variant of taking a sample, it holds that the apparatus further comprises a sample valve provided with a valve inlet, a first valve outlet and a second valve outlet, wherein the second valve outlet of the main valve is in fluid communication, for example via a seventh line, with the valve inlet of the sample valve, the first valve outlet of the sample valve is in fluid communication, for example via an eighth line, with the collecting chamber inlet and wherein the second valve outlet of the sample valve is in fluid communication with the milk line downstream of the main valve, the sample valve being configured to selectively switch between a first and second state, wherein in the first state the first valve inlet of the sample valve is in fluid communication with the first valve outlet of the sample valve so that the milk can flow through the sample valve to the sample collecting chamber, wherein in the second state the first valve inlet of the sample valve is in fluid communication with the second valve outlet of the sample valve for supplying milk which flows through the sample valve to the milk line downstream of the main valve. In particular, it holds here that the control unit switches the sample valve, when the main valve is in its second state, with such a pattern between its first and second state that the sample collecting chamber, in use, is filled with partial samples which in combination represent a picture of the total amount of milk which flows through the valve inlet.

In the above-outlined manner, the sample collecting chamber can be filled with a sample which is representative of a complete milk yield. A complete milk yield is understood to mean the complete amount of milk that is yielded by an animal in one milking. The magnitude of this sample, however, will vary depending on the magnitude of the milk yield. Also where a relatively small milk yield is concerned, the intention is for the sample that has been taken to be sufficiently large. This is why the sample that is received in the sample collecting chamber is preferably always larger than the magnitude of the volume of a desired sample, also when the milk yield of the cow is relatively small. As in this manner the magnitude of the sample is always larger than is desired, and/or varies, it holds according to a preferred embodiment of the invention that the apparatus is further provided with a sample take-up chamber whose volume is less than the sample collecting chamber's and wherein the sample collecting chamber is provided with a second sample collecting chamber outlet which, selectively, as desired, is or is not in fluid communication, for example via a tenth line in which a second switchable open-close valve is included, with an inlet of the sample take-up chamber for filling the sample take-up chamber with a predetermined amount of milk from the sample collecting chamber. In this manner, the sample take-up chamber, which can comprise a sample, can be filled with a desired volume from the sample collecting chamber which, as set out above, preferably comprises a sample that is larger than necessary. After the sample take-up chamber has been filled, the remainder of the milk that is present in the sample collecting chamber can be led back to the milk line downstream of the main valve as has been discussed above.

Preferably, it holds that the sample collecting chamber is provided with an air inlet opening for supplying air to the sample collecting chamber when milk flows from the sample collecting chamber into the sample take-up chamber. In this way, by supplying air to the sample collecting chamber, the milk present in the sample collecting chamber can be properly mixed. In particular, it may hold here that the sample take-up chamber is provided with a dispensing outlet for selectively dispensing or not dispensing the milk that is in the sample take-up chamber into an external receptacle such as a vial, type of valve such as a liquor valve, a vial of a tray which is provided with a multiplicity of vials, or a milk analysis unit, while, in particular, the dispensing outlet is formed by the sample collecting chamber outlet.

It may also be, however, that the sample take-up chamber itself can be uncoupled from the apparatus and serves as a vial in which the sample of the milk can be conveyed.

For each of the embodiments outlined hereinbefore, it is possible that the main valve is further provided with a second valve inlet, wherein in the second state the second valve inlet is in fluid communication with the first valve outlet, wherein the sample collecting chamber outlet is directly in fluid communication, for example through the second line in which the first switchable open-close valve is included, with the second valve inlet and thereby with the milk line downstream of the main valve when the main valve is in the second state, so that in the second state the excess milk can be led back from the sample collecting chamber via the main valve to the milk line. This embodiment makes it easier to incorporate the apparatus in the milk line because it is only necessary that milk from the milk line flows into the valve inlet of the main valve and milk flows from the first valve outlet to the milk line. The return flow of milk from the sample collecting chamber and/or milk flow-through unit, however, can also be supplied directly to the milk line downstream of the first valve. This, however, will as a rule require an extra adjustment, for example placement of a T-piece on the milk line. Also the return flow of milk that flows out of the second valve outlet of the sample valve may be supplied to the milk line via the second valve inlet of the main valve, or directly to the milk line. Such variants fall within the scope of the invention. The invention will presently be further elucidated on the basis of the drawing, in which:

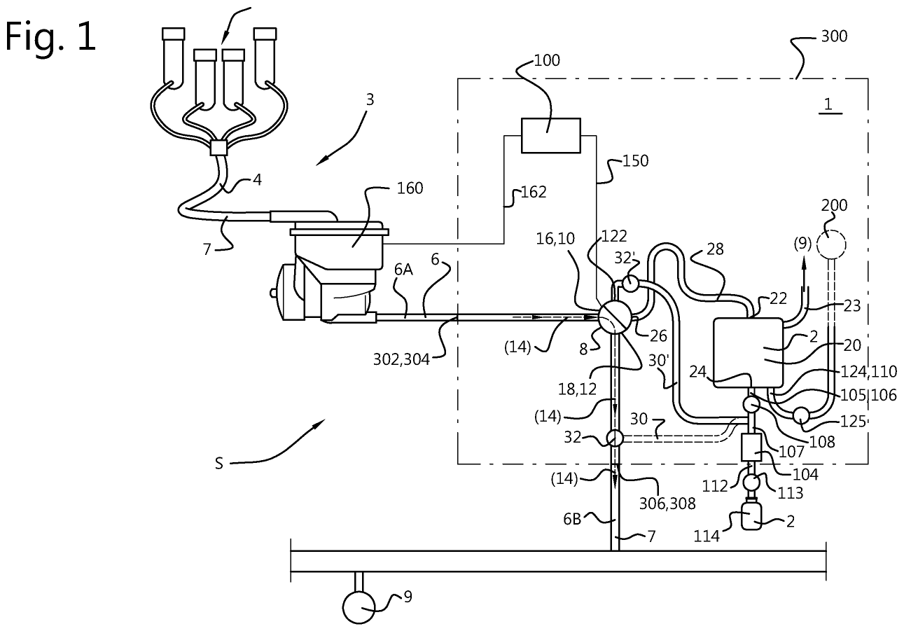

FIG. 1 shows a first embodiment of an apparatus and system according to the invention for taking a sample of milk that flows through a milk line;

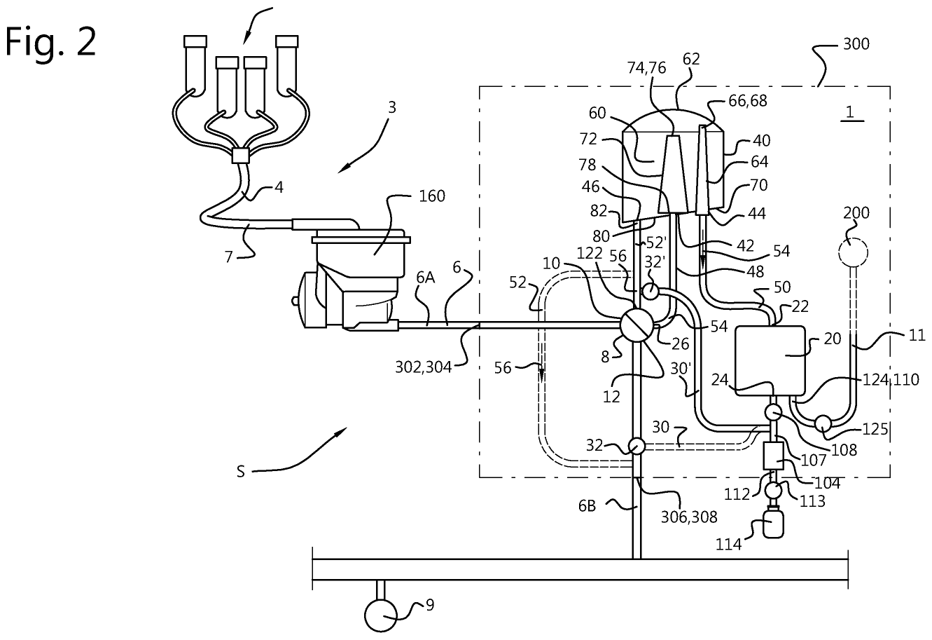

FIG. 2 shows a second embodiment of an apparatus and system according to the invention for taking a sample of milk that flows through a milk line;

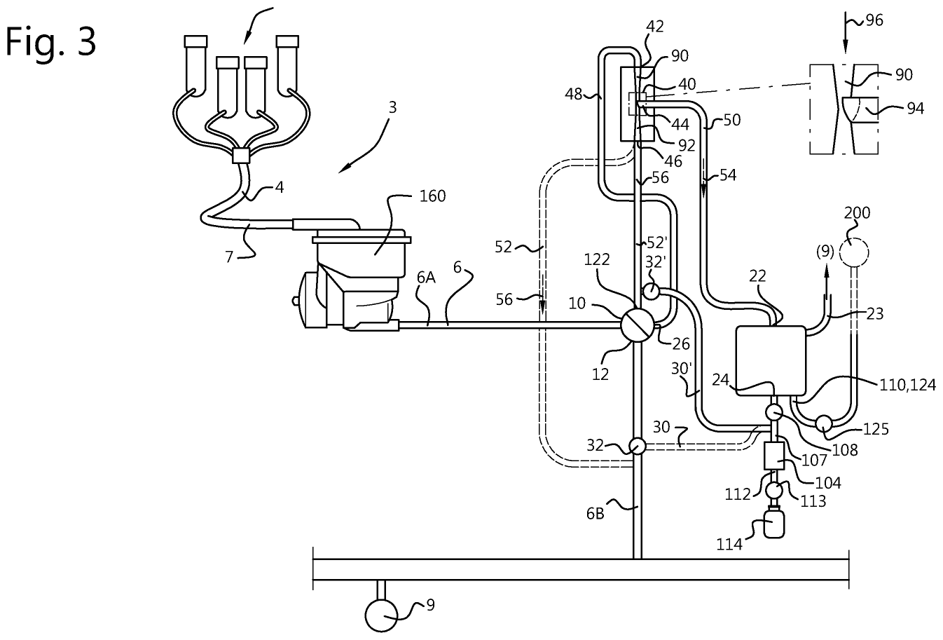

FIG. 3 shows a third embodiment of an apparatus and system according to the invention for taking a sample of milk that flows through a milk line;

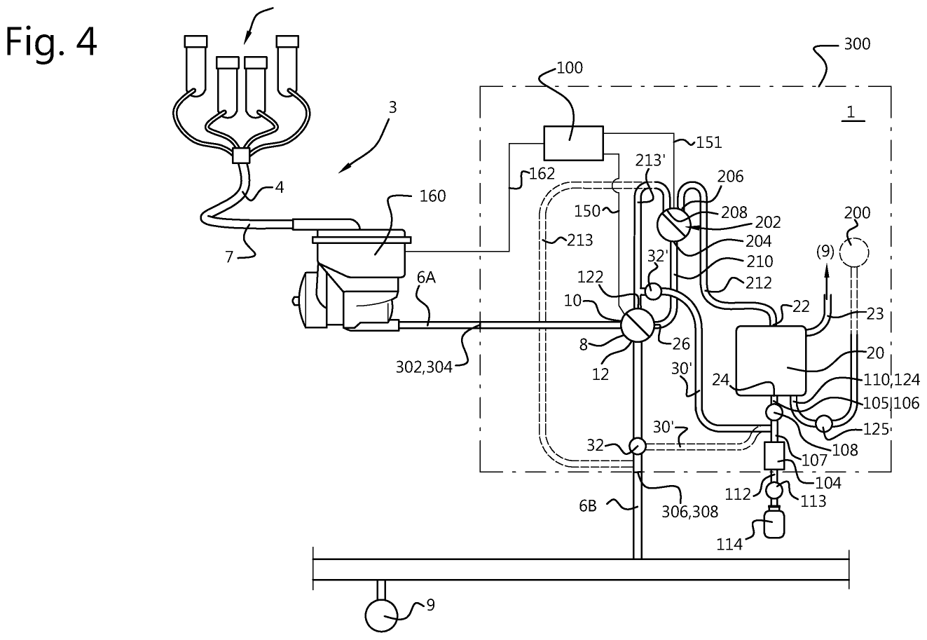

FIG. 4 shows a fourth embodiment of an apparatus and system according to the invention for taking a sample of milk that flows through a milk line;

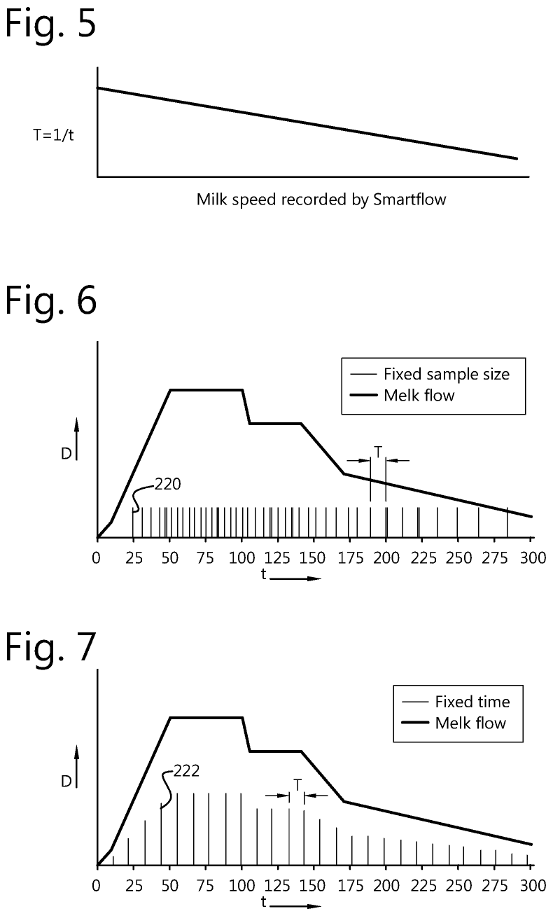

FIG. 5 shows an operation of the control unit of the apparatus according to FIG. 1;

FIG. 6 schematically shows the taking of partial samples according to a first variant according to FIG. 1;

FIG. 7 schematically shows the taking of partial samples according to a second variant according to FIG. 1;

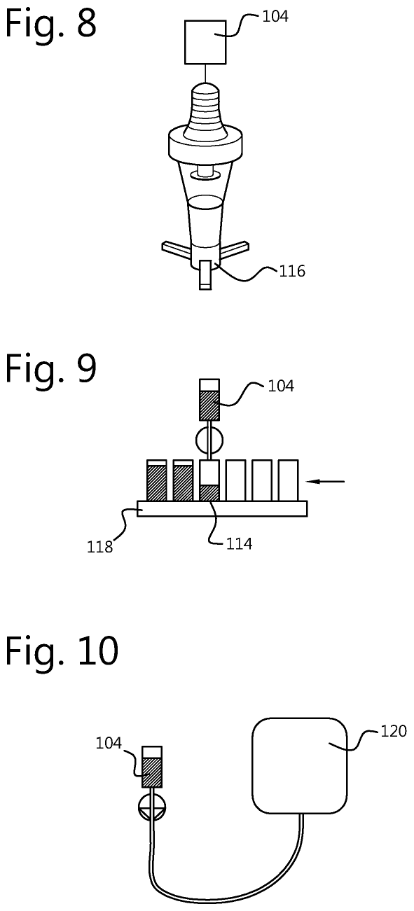

FIG. 8 shows a liquor valve of a system according to the invention which also comprises an apparatus according to the invention;

FIG. 9 shows a vial and a tray with vials of a system according to the invention which also comprises an apparatus according to the invention; and

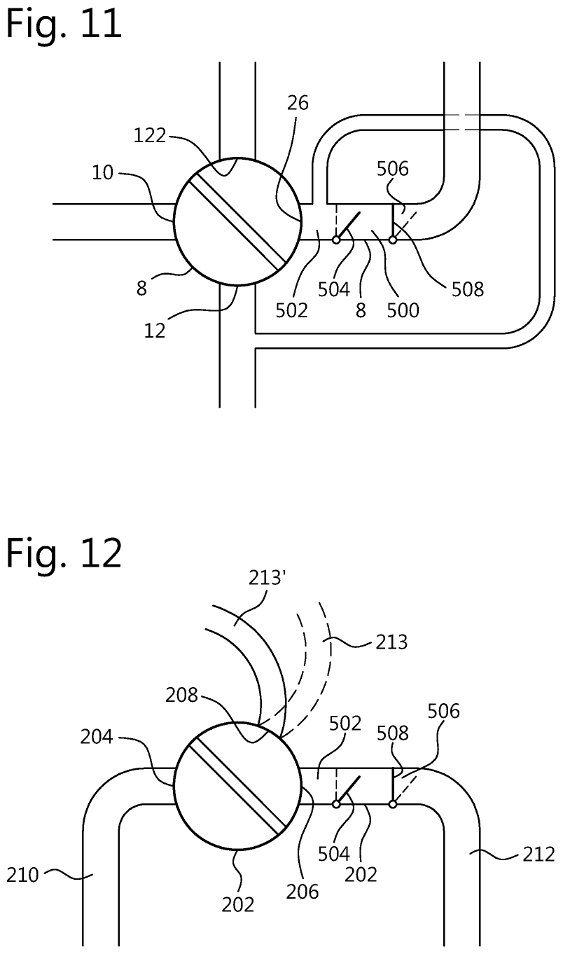

FIG. 10 shows a milk analysis unit 120 of a system according to the invention which also comprises an apparatus according to the invention; FIG. 11 shows a particular embodiment of the main valve of FIG. 1;

FIG. 12 shows a particular embodiment of the sample valve of FIG. 4; and

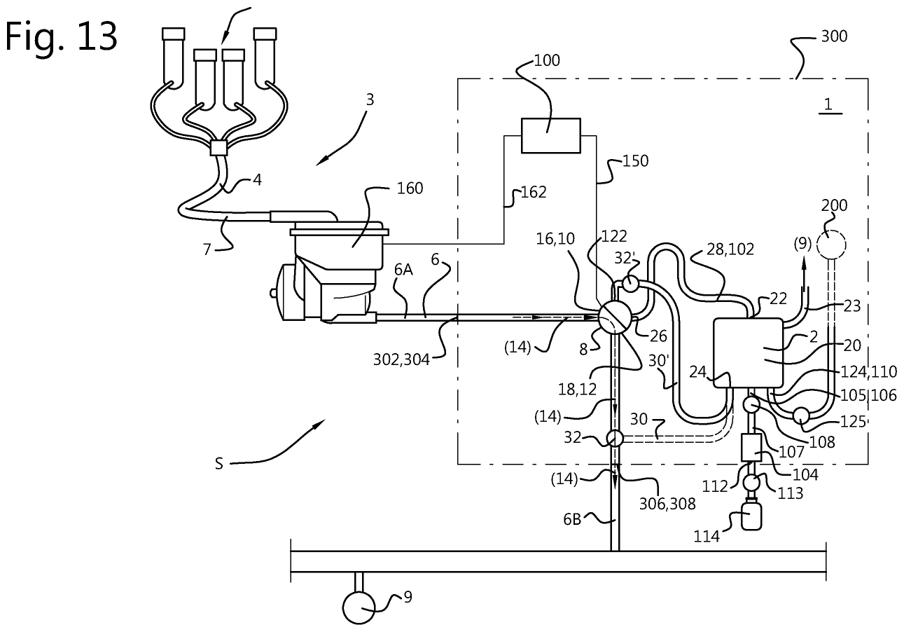

FIG. 13 shows a fifth embodiment of an apparatus and system according to the invention for taking a sample of milk that flows through a milk line.

In FIG. 1 reference S designates a system according to the invention. The system S comprises an apparatus (1) for taking a sample (2) of milk (4) that flows through a milk line (6). The milk which flows through the milk line originates from a milking device (3) known per se, which comprises a set of teat cups (5), a milk hose (7) and a vacuum pump (9). The apparatus includes a main valve (8) comprising a first valve inlet (10) and a first valve outlet (12). The apparatus is configured for including the main valve in a milk flow path through the milk line by placing the first valve inlet in fluid communication with a first open end (16) of the milk line and placing the first valve outlet in fluid communication with a second open end (18) of the milk line. In the milk flow path the first open end is located upstream with respect to the second open end. In effect, the milk line has been cut open for obtaining the first (16) and second (18) open ends. The apparatus further comprises a sample collecting chamber (20) for receiving the sample of the milk, the sample collecting chamber being provided with a sample collecting chamber inlet (22) and a sample collecting chamber outlet (24) which is provided with a second switchable open-close valve (108). The apparatus further comprises a fluid communication (23) which is configured to be connected with a vacuum pump (9) for lowering the pressure in the sample collecting chamber. To this end, the sample collecting chamber in this example is connected through a schematically shown line (23) with the vacuum pump (9) for, just as in the milk line (6), lowering the pressure in the sample collecting chamber (20). The main valve further comprises a second valve outlet (26), the second valve outlet (26) being in fluid communication, in this example through at least a first line (28), with the sample collecting chamber inlet (22). The main valve is configured to selectively switch between a first and second state, where in the first state the first valve inlet (10) is in fluid communication with the first valve outlet (12) so that the milk can flow through the milk line without a sample being taken. In other words, the milk flows from milk line section (6A) directly via the main valve into milk line section (6B). In the second state, the first valve inlet (10) is in fluid communication with the second valve outlet (26) for supplying milk from the milk line (from milk line section (6A)) to the sample collecting chamber. The apparatus is further configured for selectively, when the second open-close valve (108) is open, placing or not placing the sample collecting chamber outlet (24) in fluid communication, for example through a second line (30) in which a first switchable open-close valve (32) is included, with the milk line (with milk line section (6B)) downstream of the main valve for leading back to the milk line (6) excess milk of a milk sample which is in the sample collecting chamber. In the drawing, the second line (30) is represented with a broken line, and in this variant the line (30′) and the open-close valve (32′) can be thought away. In this example, it holds furthermore that the apparatus is in fluid communication with the sample take-up chamber (104) whose volume is smaller than the sample collecting chamber's. The sample take-up chamber is provided with an inlet (107) which is in fluid communication with the outlet of the valve (108). The sample take-up chamber can be a fixed part of the apparatus or a chamber which is used once to be filled with a milk sample. In this example, it is a fixed part of the apparatus. The apparatus described up to this point works as follows.

When milk flows through the milk line (6), the main valve can take up the first state. In that case, the milk flows via the valve directly from the milk line section (6A) into the milk line section (6B). When, however, it is desired to take a sample of the milk that flows through the milk line (6), the main valve is brought into the second state. The valve (108) is closed. In that case, milk will flow from the milk line section (6A) via the main valve (8) and via the line (28) into the sample collecting chamber (20). This sample collecting chamber (20) will be further filled with a sample of the milk. After this has been done, the valve (108) is opened for filling the sample take-up chamber (104). When the sample take-up chamber (104) has been filled, it comprises a sample of the milk having a predetermined volume. The sample collecting chamber (20) then will not be empty yet and can empty further by opening of the valve (32′). The consequence is that the milk sample's excess milk present in the collecting chamber (20) is led back to the milk line (6). The valves (108) and (32′) may also be opened simultaneously.

Furthermore, it holds in this example that the sample collecting chamber is provided with an air inlet opening (110) for supplying air to the sample collecting chamber when milk flows from the sample collecting chamber into the sample take-up chamber. In this example, it holds furthermore that the sample collecting chamber is provided with an air supply opening (124), configured to be opened and closed by means of an air valve (125) in order to, in a settable manner, supply or not supply air to the sample collecting chamber, the air supply opening being located at an underside of the sample collecting chamber. In this manner, the sample collecting chamber is configured for mixing of the milk in the sample collecting chamber by sufficient air supply. In this example, the air supply openings (124) and the air inlet opening (110) are configured, combined, as one opening and have a double function. It is also possible to provide the air inlet opening at an upper side of the sample collecting chamber for the purpose of the emptying of the sample collecting chamber and to provide the air supply opening (124), by contrast, in the bottom for the purpose of the mixing of the milk in the sample collecting chamber. It is hence also possible that the sample collecting chamber is provided at its upper side with an opening for supplying air to the sample collecting chamber when the sample collecting chamber, at least in part, empties into the sample take-up chamber (104) and is provided with a second air supply opening (124) which is at an underside of a sample collecting chamber for the supply of air to the sample collecting chamber from the underside of the sample collecting chamber, to bring about the mixing of the milk which is in the sample collecting chamber.

In this example, it holds furthermore that the sample take-up chamber (104) is provided with a dispensing outlet (112) and a valve (113) so as to be able upon opening of the valve (113) to dispense the milk that is present in the sample take-up chamber via the outlet (112) into an external receptacle such as a vial (114) (see FIG. 9), valve of a type such as a liquor valve (116) (see FIG. 8), a vial of a tray (118) which is provided with a multiplicity of vials (see FIG. 9) or a milk analysis unit (120) (FIG. 10). The valve (116) works as known in the catering industry: by pushing up the valve at its underside with the vial, the valve (116) will open and the vial will be filled.

Presently, with reference to FIG. 1, a highly advanced embodiment of the invention will be elucidated. To this end, the apparatus according to FIG. 1 is provided with a control unit (100) for controlling the main valve (8) from its first state to its second state and vice versa. Control signals are sent via the electrical line (150) from the control unit to the main valve (8). The apparatus is further provided with a sensor (160) (schematically indicated) with which the flow of the milk through the milk line, in this example in the milk line section (6A), can be measured. The information which is obtained with the aid of the sensor is supplied to the control unit (100) via line (162) or wirelessly (so that line 162 can be omitted). The operation of the apparatus is as follows.

When no samples need to be taken, the control unit (100) sets the main valve (8) in the first state so that milk flows via the valve (8) directly from the milk line section (6A) to the milk line section (6B). When, however, a sample is to be taken from the milk which flows through the milk line (6), operation is as follows.

First of all, the valve (32), which is also controlled by the control unit (100) (the control line running from the control unit (100) to the valve (32) is not shown), is closed. Also, the valve (108) is closed by the control unit (100)) (the control line running from the control unit (100) to the valve (108) is not shown). The control unit (100) will control the main valve (8) with such a pattern from its first state to its second state and vice versa that the sample collecting chamber (20), in use, is filled with partial samples which in combination represent a picture of the total amount of milk that flows through the valve inlet of the main valve. Due to the main valve being switched alternately between the first and second state by the control unit, this also means that milk that flows through the milk line section (6A) is supplied alternately to the milk line (6B) or to the sample collecting chamber (20). Due to the partial samples being taken at different points of time, for example spread in time over the total milk yield of a cow being milked, the partial samples together, when received in sample collecting chamber (20), give a good picture of the milk which has flowed through the milk line, also when properties of the milk vary during the period in which the partial samples are taken. Indeed, when, for example, at the start of the milk yield of a milking cow the fat content in the milk is low whilst being high at the end of the milk yield, this will still be established in that partial samples of the milk being yielded by the cow are taken spread in time.

In particular, it holds in the present embodiment that the control unit controls the main valve from its first state to its second state and vice versa with such a pattern that the sample collecting chamber, in use, is filled with partial samples which in combination represent a picture of the total amount of milk that has flowed through the valve inlet of the main valve.

According to a first variant, the control unit to that end measures with the aid of the sensor (160) the flow of the milk stream through the valve inlet (8) and proceeds to control the valve unit such that the sample collecting chamber, in use, is filled with partial samples having a same volume and with a spread in the time in which the partial samples are taken, while the frequency at which the partial samples are taken is proportional to the magnitude of the flow of the milk stream through the valve inlet. That the partial samples each have a same volume is achieved in that the main valve (8) in each case is brought into the second state for a time interval of a fixed length. The time duration of the first state, however, is variable and is inversely proportional to the magnitude of the flow of the milk stream through the valve inlet. If the magnitude of the flow of the milk stream through the valve inlet increases, the time duration in which the valve remains in a first state hence decreases. Put differently, the frequency at which samples are taken, that is, the frequency at which the second states occur in time increases when the flow of a milk stream increases, and vice versa.

FIG. 5 plots the relation between T and the flow measured by the sensor. T is here defined as the time lapse between the middle of a period in which the valve is in the first state and the middle of a next period in which the valve is in the first state again. The frequency mentioned is therefore equal to 1/T.

Further explanations of the above are set out with reference to FIG. 6. In FIG. 6, by way of example the period T is indicated. Further shown in FIG. 6 is the course in time t of a flow D measured with the sensor. Further, with upstanding lines (220) the periods are indicated in which the valve is in the second state. The periods between the upstanding lines are the periods in which the valve is in the first state. Because in the example of FIG. 6 the magnitude of the partial samples, that is, the volume of the partial samples, is the same in each case, the respective lines are equally wide. The distance between the lines, however, varies depending on the measured flow. The magnitude of each of the partial samples is the same, due to the main valve (8) being provided to that end with a special sample filling chamber (500), see FIG. 11. The sample filling chamber is downstream of the second valve outlet (26). The sample filling chamber has an inlet (502) which can be opened and closed with a valve (504) controllable with the control unit (100), and an outlet (506) which can be opened and closed with a valve (508) controllable with the control unit (100). When the main valve (8) is in its second state, the valve (504) is opened and the valve (508) is closed. The sample filling chamber (500) then fills with milk. After a sufficiently long time, the control unit (100) will close the valve (504). This length of time has been chosen to be so long that at current rates of flow of the milk that flows through the milk line the sample filling chamber is completely filled. The sample filling chamber (500) is now completely filled with the milk. This milk is the partial sample having a fixed predetermined volume. Because the main valve is still in the second state, the milk will now flow from the second outlet via a line (510) to the milk line section (6B). Next, the control unit (100) opens the valve (508) so that the partial sample will flow to the sample collecting chamber (20). After this, the control unit (100) will bring the main valve (8) into its first state as discussed above and the milk flows directly from milk line section (6A) to milk line section (6B) without a sample being taken (in the period between the vertical lines in FIG. 6). The valve (504) is opened again by the control unit (100) and the valve (508) is closed by the control unit (100) so that thereupon the main valve can again be brought into the second state by the control unit for taking a next partial sample, analogously to what has been discussed above regarding the previous partial sample.

According to another variant, however, it is also possible that the control unit (100) controls the main valve (8) with such a pattern from its first state to its second state and vice versa that the sample collecting chamber, in use, is filled with partial samples having volumes that are proportional to the magnitude of the flow of the milk stream through the valve inlet, with the partial samples being taken at a fixed frequency. It holds, then, that the magnitude of a volume in each case is proportional to the magnitude of the flow of the milk stream through the valve inlet, with the partial samples being taken at a fixed frequency.

An example of this is shown in FIG. 7. In FIG. 7, upstanding lines (222) denote the presence of the second state. The periods between the upstanding lines (222) correspond to periods in which the main valve is in the first state. In this example, the period T between two successive lines is constant. Due to the lines also having a fixed width B sec, it also holds that the period T running from halfway a line up to halfway a next line is constant. It holds, therefore, that the partial samples are taken at a fixed frequency. The volume of the partial samples, however, is variable in the sense that they are proportional to the magnitude of the flow. This is inherent to the system itself: the magnitude of the partial sample is equal to the magnitude of the flow times B. Next, the valve (125) is opened by the control unit (100) (the control line running from the control unit (100) to the valve (125) is not shown), whereby air is supplied to the opening for the mixing of milk which is collected in the sample collecting chamber.

Presently, with reference to FIG. 2, a second embodiment of the apparatus according to the invention will be discussed. Parts corresponding to FIG. 1 are provided with a same reference numeral. With respect to FIG. 1, the apparatus is further provided with a milk flow-through unit (40). The milk flow-through unit (40) is provided with a milk flow-through unit inlet (42), a first milk flow-through unit outlet (44) and a second milk flow-through unit outlet (46). In this example, the line (23) can be omitted because via the milk flow-through unit outlet (44) the pressure in the sample collecting chamber (2) is lowered as in the milk line (6). The second valve outlet (26) is in fluid communication, in this example via a third line (48), with the milk flow-through unit inlet (42). The first milk flow-through unit outlet (44) is in fluid communication, in this example via a fourth line (50), with the sample collecting chamber inlet (22) so that the fluid communication between the second valve outlet (26) and the sample collecting chamber inlet (22) extends through the milk flow-through unit (40). The second milk flow-through unit outlet (46) is in fluid communication, in this example through a fifth line (52), with the milk line downstream of the main valve. The lines indicated with reference numerals (52′) and (30′) as well as the valve (32′) can be thought away. The milk flow-through unit (40) comprises a chamber (60) with a ceiling (62), a first upstanding line section (64) which comprises an open end (66) at its upper side (68) and of which an open underside (70) is connected with the first milk flow-through unit outlet (44). Further, the milk flow-through unit (40) comprises a second upstanding line section (72) which comprises an open end (74) at its upper side (76) and of which an open underside (78) is connected with the milk flow-through unit inlet (42). The milk flow-through unit (40) furthermore comprises a bottom (80) with an outflow opening (82) which is connected with the second milk flow-through unit outlet (46). The milk flow-through unit is so configured that a milk stream which is supplied to the milk flow-through unit inlet (42) is, inside the chamber, directed upwards and via the second upstanding line section (72) spouts against the ceiling (62) to then leave the milk flow-through unit respectively via the open end (66) at the upper side (68) of the first upstanding line section (64), the rest of the first upstanding line section and the first milk flow-through unit outlet (44) as a first milk stream (54) and via the outflow opening (82) and the second milk flow-through unit outlet (46) as a second milk stream (56). By tuning the size of the open end (66) and the size of the outflow opening to each other, the ratio of the flow of the first milk stream and the flow of the second milk stream can be defined. It holds, then, that the milk flow-through unit is so configured that a milk stream which is supplied to the milk flow-through unit inlet (42) of the milk flow-through unit splits up into a first milk stream (54) and a second milk stream (56) which leave the milk flow-through unit respectively via the first milk flow-through unit outlet (44) and the second milk flow-through unit outlet (46). The flow of the first and the flow of the second milk stream have a predetermined ratio so that, in use, when the main valve is in the second state, a portion of the milk that flows through the second valve outlet (26) is supplied to the sample collecting chamber by way of the first milk stream and a portion of the milk that flows through the second valve outlet (26) is supplied to the milk line downstream of the main valve (to milk line section (6B)) by way of the second milk stream for return of this milk to the milk line. The magnitude of the flow of the second milk stream is, for example, 1.5-5%, in particular 2%, of the magnitude of the flow of the milk stream to the first valve inlet of the main valve. The operation of the embodiment of FIG. 2 as described up to this point is as follows.

The main valve is brought into the first state when no sample needs to be taken. This can be done manually or with the control unit. The control unit in this variant is therefore not necessary for controlling the valves, but, of course, it may be so. The control unit is not shown in this example, so that manual operation is assumed. When, however, a sample is to be taken from the milk that flows through the milk line (6), for example a sample that is obtained from an at least substantially complete milk yield of an animal when it is milked, the main valve is brought into the second state. The valve (108) is closed here. Also the valve (32) is closed here. The result is that with the aid of the milk flow-through unit (40), the milk stream which is supplied to the valve inlet of the main valve is split up into, respectively, the first milk stream (54) and the second milk stream (56). The result is that the sample collecting chamber (20) is slowly filled by the first milk stream. The second milk stream (56) is supplied via the line (52) to the milk line (6) downstream of the main milk valve (8). This procedure can be continued throughout the process of milking a cow. The milk that is collected in the sample collecting chamber (20) represents a good sample of the total milk given by an animal. Depending on the magnitude of a milk yield of the animal, more or less milk will be collected in the sample collecting chamber (20) during a complete milking of the animal. Afterwards, air is supplied to the air inlet opening (10) with the aid of the air pump (200). The valve (125) is opened so that by means of the air the milk is properly mixed in the sample collecting chamber (20). When milking is finished, the valve (108) can be opened to fill the sample take-up chamber (104) with a predetermined amount of the milk. Thus, a sample has been obtained having a predetermined volume, which, moreover, is a good representative of the total milk given by the animal. The valve (32) is opened so that the surplus of the milk can overflow from the sample collecting chamber (20) into the milk line, that is, into the milk line section (6B). When the sample take-up chamber (104) has been filled, the valves (108) and (32) can be closed. The main valve can be brought into the first state again when there is no intention to take further samples.

Presently, a third embodiment of the invention will be discussed with reference to FIG. 3 where parts corresponding to FIGS. 1 and 2 have been provided with a same reference numeral as in these figures, but where for clarity's sake a number of reference numerals have been omitted. Just as with FIG. 2, the apparatus comprises a milk flow-through unit (40) provided with a milk flow-through unit inlet (42), a first milk flow-through unit outlet (44) and a second milk flow-through unit outlet (46). The second valve outlet (26) is in fluid communication, in this example through a third line (48), with the milk flow-through unit inlet (42). The first milk flow-through unit outlet (44) is in fluid communication, in this example through a fourth line (50), with the sample collecting chamber inlet (22) so that the fluid connection between the second valve outlet (26) and the sample collecting chamber inlet (22) extends through the milk flow-through unit (40). The second milk flow-through unit outlet (46) is in fluid communication, in this example through a fifth line (52), with the milk line (milk line section (6B)) downstream of the main valve. Lines (52′) and (30′) as well as the valve (32′) can be thought away again.

The milk flow-through unit (40) comprises a chamber (90) provided with the milk flow-through unit inlet (42), the first milk flow-through unit outlet (44) and the second milk flow-through unit outlet (46). The milk flow-through unit inlet (42) and the second milk flow-through unit outlet (46) are opposite to each other. The first milk flow-through unit outlet (44) is in between the milk flow-through unit inlet and the second milk flow-through unit outlet. The milk flow-through unit is so configured that a milk stream which is supplied to the milk flow-through unit inlet (42) leaves the milk flow-through unit via the first milk flow-through unit outlet (44) in a first milk stream (54) and via the second milk flow-through unit outlet in a second milk stream (56). The milk flow-through unit comprises to this end a channel (92), formed by the chamber (90), which extends, preferably in vertical direction, from the first milk flow-through unit inlet (42) to the second milk flow-through unit outlet (46). The channel is provided, at the location of the first milk flow-through unit outlet, with a milk stream deflecting element (94), for deflecting a part of the milk stream flowing in the direction from the milk flow-through unit inlet (42) to the first milk flow-through unit outlet (44). The milk deflecting element is in the form of a scoop or trough which, from a sidewall of the channel, projects into the channel (90), that is, into the channel (92), for example approximately as far as the middle of the channel (92). This ‘trough’ or ‘scoop’ terminates in the first milk flow-through unit outlet (44).

The milk stream deflecting element (94) is located approximately halfway the channel, viewed in a flow-through direction (96) of the channel. By tuning the size of the milk deflecting element (42) and/or the size of a flow-through passage area of the first milk flow-through unit outlet (44) on the one hand and a flow-through passage area of the second milk flow-through unit outlet (46) to each other, the ratio of the magnitude of the flow of the first milk stream to the magnitude of the flow of the second milk stream can be attuned.

The magnitude of the flow of the second milk stream is, for example, 1.5-5%, in particular 2%, of the magnitude of the flow of the milk stream to the first valve inlet of the main valve.

It holds, then, just as with FIG. 2, that the milk flow-through unit is so configured that a milk stream which is supplied to the milk flow-through unit inlet (42) of the milk flow-through unit splits up into the first milk stream (54) and the second milk stream (56) which leave the milk flow-through unit respectively via the first milk flow-through unit outlet (44) and the second milk flow-through unit outlet (46). It holds here that the flow of the first and second milk stream has a predetermined ratio so that, in use, when the main valve is in the second state, a portion of the milk that flows through the second valve outlet (26) is supplied to the sample collecting chamber by way of the first milk stream and a portion of the milk that flows through the second valve outlet (26) is supplied to the milk line downstream of the main valve by way of the second milk stream for return of this milk to the milk line. The first and second milk stream are generated when the main valve is in the second state.

The operation of this embodiment is as follows. The main valve is brought into the first state when no sample needs to be taken. When, however, a sample is to be taken from milk flowing through the milk line (6), for example a sample that is obtained from an at least substantially complete milk yield of an animal when it is milked, the main valve is brought into the second state. The valve (108) is closed here. Also the valve (32) is closed here. The result is that with the aid of the milk flow-through unit (40), the milk stream which is supplied to the valve inlet of the main valve is split up into, respectively, the first milk stream (54) and the second milk stream (56). The result is that the sample collecting chamber (20) is slowly filled by the first milk stream. The second milk stream (56) is supplied via the line (52) to the milk line (6) downstream of the main milk valve (8). This procedure can be continued throughout the process of milking a cow. The milk that is collected in the sample collecting chamber (20) represents a good sample of the total milk given by an animal. Depending on the magnitude of a milk yield of the animal, more or less milk will be collected in the sample collecting chamber (20) during a complete milking of the animal. When milking is finished, air is supplied to the air inlet opening (10). The valve (125) is opened so that by means of the air the milk is properly mixed in the sample collecting chamber (20). Afterwards, the valves (108) and (32) can be opened to fill the sample take-up chamber (104) with a predetermined amount of the milk and to allow the surplus of the milk to flow into the milk line via line (30). Thus, a sample has been obtained having a predetermined volume, which, moreover, is a good representative of the total milk which has been given by the animal. When the sample take-up chamber (104) has emptied, the valves (108) and (32) can be closed. The main valve can be brought into the first state again when there is no intention to take further samples.

Presently, on the basis of FIG. 4, a fourth embodiment of the invention will be discussed which is closely related to the embodiment according to FIG. 1.

In FIGS. 1 and 4, mutually corresponding parts have been provided with a same reference numeral, but some reference numerals have been omitted for reasons of clarity.

The apparatus according to FIG. 4 further comprises a sample valve (202) provided with a valve inlet (204), a first valve outlet (206) and a second valve outlet (208). The second valve outlet (26) of the main valve (8) is in fluid communication, in this example via a seventh line (210), with the valve inlet (204) of the sample valve. Furthermore, the first valve outlet (206) of the sample valve is in fluid communication, in this example via an eighth line (212), with the sample collecting chamber inlet. The second valve outlet (208) of the sample valve is in fluid communication, in this example via a ninth line (213), with the milk line downstream of the main valve. The sample valve is configured to selectively switch between a first and second state wherein in the first state the first valve inlet of the sample valve is in fluid communication with the first valve outlet of the sample valve so that the milk can flow through the sample valve to the sample collecting chamber, and in the second state the first valve inlet of the sample valve is in fluid communication with the second valve outlet of the sample valve for supplying milk which flows through the sample valve to the milk line downstream of the main valve.

The apparatus furthermore comprises the control unit (100) and the sensor (160) which are connected with each other through the electrical line (162). In this example, the control unit (100) controls the sample valve (204) via the electrical line (151), while it is also possible that the control unit (100), via the electrical line (150), controls the main valve (8) and possibly also one or more of the other valves. The operation of the apparatus described up to this point is as follows.

The main valve (8) is brought into the first state, for example by the control unit (100), when no sample needs to be taken. When, however, a sample is to be taken, the main valve (8) is brought into the second state. This can be carried out, for example, with the aid of the control unit (100) or manually. The result is that milk that flows through the milk line section (6A) flows via the main valve (8) and the seventh line (210) to the valve inlet of the sample valve (202).

The sample valve is now brought alternately into the first and the second state by the control unit (100), all this entirely analogously to what has been discussed for the main valve in relation to the embodiment of FIG. 1. It holds, then, that the control unit switches a sample valve with such a pattern between its first and second state that the sample collecting chamber (20) is filled with partial samples which in combination represent a picture of the total amount of milk flowing through the valve inlet of the sample valve. Entirely analogously to what has been discussed in relation to FIG. 1, there are two possibilities. According to the first possibility, the control unit switches the sample valve with such a pattern between its first and second state that the sample collecting chamber, in use, is filled with partial samples having a same volume and with a spread in the time in which the partial samples are taken of which the frequency is proportional to the magnitude of the flow of a milk stream through the valve inlet. The magnitude of each of the partial samples is the same, due to the sample valve (8) in this variant being provided to that end with the special sample filling chamber (500), see FIG. 12. The sample filling chamber is located downstream of the first valve outlet (206). The sample filling chamber has the inlet (502) which can be opened and closed under the control of the control unit (100) with the controllable valve (504) and the outlet (506) which can be opened and closed with the valve (508) controllable with the control unit (100). When the main valve (8) is in its second state and the sample valve is in its first state, the valve (504) is opened and the valve (508) is closed. The sample filling chamber (500) then fills with milk. After a sufficiently long time, the control unit (100) will close the valve (504). This length of time has been chosen to be so long that at current rates of flow of the milk flowing through the milk line, the sample filling chamber is completely filled. The sample filling chamber (500) is now completely filled with the milk. This milk is the partial sample having a fixed predetermined volume. Because the main valve is still in the second state, the milk will now flow from the first outlet via a line (510) to the milk line section (6B). Then the control unit (100) opens the valve (508) so that the partial sample will flow to the sample collecting chamber (20). After this, the control unit will bring the sample valve into the second state and the milk will flow via line (213) to the milk line section (6B). After this, the control unit will bring the main valve (8) into its first state as has been discussed above and the milk flows directly from milk line section (6A) to milk line section (6B) without a sample being taken (in the period between the vertical lines in FIG. 6). The valve (504) is opened again by the control unit (100) and the valve (508) is closed by the control unit (100) so that thereupon the main valve can again be brought into the second state by the control unit and the sample valve into the first state by the control unit for taking a next partial sample, analogously to what has been discussed hereinbefore for the previous partial sample.

According to a second variant, the control unit switches the sample valve with such a pattern between its first and second state that the sample collecting chamber, in use, is filled with partial samples having volumes that are proportional to the magnitude of the flow of the milk stream through the valve inlet of the main valve, the partial samples being taken at a fixed frequency. Here too, it holds that, preferably, the partial samples together constitute 1.5-5% of the milk that has been supplied to the valve inlet of the sample valve. In use, the sample collecting chamber (20) will again be slowly filled with the partial samples. Because the partial samples together can constitute 1.5-5%, in particular 2%, of the total milk yield of an animal, in the sample collecting chamber always sufficient milk will be formed to be able to fill the sample take-up chamber (104) completely with a predetermined amount of milk. This is then done again by opening the valves (108) and (32). After the sample take-up chamber (104) has been filled with the predetermined amount of milk, the surplus flows over to the milk line (6B) and the valves (108) and (32) can be closed again. Obviously, during the filling of the sample collecting chamber, the valve (108) and possibly also the valve (32) were closed. After filling, the air valve (125) is opened so that again air can be supplied to the sample collecting chamber (20) for the proper mixing of the sample so that also the sample that is eventually taken up in the sample take-up chamber is a good representative of the total milk yield of an animal. In all of the above-outlined variants, the part that has been indicated within the chain-dotted framework (300) can belong to the apparatus. This means that a milk line, for the purpose of installing the apparatus, only needs to be cut open at one point. The milk line can then be connected at the point (302) with an inlet (304) of the apparatus, and be connected at the point (306) with an outlet (308) of the apparatus. It holds, then, that the apparatus is provided with an inlet (304) which is in fluid communication with a first valve inlet of the main valve, and an outlet (308) which is in fluid communication with the first valve outlet, while for the operation of the apparatus an existing milk line needs to be cut open only at one point to connect the apparatus with the milk line, while in particular milk which is in the apparatus and which does not belong or is not going to belong to a sample can flow back to the milk line via the outlet.

The invention is not limited in any way to the above-outlined embodiments. Thus, it holds for each of the embodiments that the main valve may further be provided with a second valve inlet (122). It holds then that in the second state the second valve inlet (122) is in fluid communication with the first valve outlet (12). In the variant of FIG. 1 the line (30) and valve (32) may then be replaced by the line (30′) and valve (32′). The consequence is that return milk, as a result of the opening of a valve (108′), can be supplied to the second valve inlet of the main valve when this valve is in the second state. This milk will then flow via the valve (8) to the milk line section (6B). An advantage is that this variant is somewhat simpler. It holds, then, in this variant, that the second milk flow-through unit outlet (46) is directly in fluid communication, for example through the fifth line (52), with the second valve inlet (122) for, in the second state of the main valve, supplying the second milk stream via the main valve to the milk line downstream of the main valve.

In the variant according to FIG. 2, it also holds that the line (30) and valve (32) can be omitted and be replaced by the line (30′) with the valve (32′). Likewise, the line (52) may be replaced by an eleventh line (52′) which extends from the milk flow-through unit outlet (46) to the second valve inlet (122). In this case, too, then, the milk that is to be returned is supplied from the sample collecting chamber (20) to the second valve inlet (122). It holds here, then, that the second milk flow-through unit outlet (46) is directly in fluid communication, through the eleventh line (52′), with the second valve inlet (122) for, in the second state of the main valve, supplying the second milk stream via the main valve to the milk line downstream of the main valve.

A similar thing then also applies to FIG. 3. For FIG. 3 too, it holds that the line (30) with the valve (32) can be replaced by the line (30′) with the valve (32′). Milk that flows through the line (30′) when the valve (32′) is open is then likewise supplied to the second valve inlet of the main valve, to flow via the main valve to the milk line section (6B). Also, the line (52) may be replaced by the line (52′). This means then that milk flowing out of the second milk flow-through unit outlet (46) can flow via the line (32′) to the second inlet of the main milk valve so that this milk, when the main valve during the taking of a sample is in the second state, can flow and return to the milk line section (6B).

For the apparatus according to FIG. 4, it also holds that the line (30) with the valve (32) can be replaced by the line (30′) with the valve (32′). Furthermore, the line (213) may be replaced by the line (213′), the line (213′) extending from the second valve outlet of the sample valve to the second valve inlet of the main valve for leading milk that does not need to be supplied to the sample collecting chamber (20) back to the milk line via the main valve (8).

In all of the examples given, it holds that the second line (30, 30′) and the tenth line (106) are each connected with the outlet of the second switchable open-close valve (108). In all of the embodiments, however, it is also possible that the sample collecting chamber (20) is provided with a separate outlet with which the second line (30, 30′) is connected. This separate outlet can then be designated as a first sample collecting chamber outlet (24) of the sample collecting chamber (20), and the outlet (105) can then be designated as a second sample collecting chamber outlet (105) of the sample collecting chamber (20). This is shown in FIG. 13, which is a variant of FIG. 1. Thus, for example, first the valve (108) is opened to fill the sample take-up chamber (104) from the sample collecting chamber. After that, the valve (108) can be closed and the valve (32) or (32′) be opened to allow the excess milk to flow out of the sample collecting chamber back into the milk line (6). This manner of returning milk may be applied entirely analogously in the variants according to FIGS. 2-4. In effect, in the variants according to FIGS. 1-4, the first sample collecting chamber outlet (24) of the sample collecting chamber (20) and the second sample collecting chamber outlet (105) of the sample collecting chamber (20) have been combined into one outlet. However, this is not requisite.

Such variants are each understood to fall within the scope of the invention.

1. An apparatus for taking a sample of milk that flows through a milk line, wherein the apparatus comprises a main valve comprising a first valve inlet and a first valve outlet,
wherein the apparatus is configured for including the main valve in a milk flow path of the milk line by placing the first valve inlet in fluid communication with a first open end of the milk line and placing the first valve outlet in fluid communication with a second open end of the milk line, the first open end being located upstream of the second open end in the milk flow path,
wherein the apparatus further comprises a sample collecting chamber for receiving the sample of the milk, the sample collecting chamber comprising:
a sample collecting chamber inlet, and
at least one sample collecting chamber outlet;
wherein the main valve further comprises a second valve outlet in fluid communication with the sample collecting chamber inlet,
wherein the main valve is configured to selectively switch between a first state and a second state,
wherein in the first state the first valve inlet is in fluid communication with the first valve outlet so that the milk can flow through the milk line without a sample being taken, and
wherein in the second state the first valve inlet is in fluid communication with the second valve outlet for supplying milk from the milk line to the sample collecting chamber, and
wherein the apparatus is configured to selectively place or not place the sample collecting chamber outlet in fluid communication with the milk line at a position downstream of the main valve for leading back to the milk line excess milk of a milk sample that is in the sample collecting chamber.

2. The apparatus according to claim 1, wherein the at least one sample collecting chamber outlet can be brought into fluid communication through a second line with the milk line for leading back the excess milk.

3. The apparatus according to claim 2, wherein a first switchable open-close valve is included in the second line.

4. The apparatus according to claim 1, wherein the apparatus further comprises a milk flow-through unit comprising:
a milk flow-through unit inlet,
a first milk flow-through unit outlet, and
a second milk flow-through unit outlet;
wherein the second valve outlet is in fluid communication, with the milk flow-through unit inlet and the first milk flow-through unit outlet is in fluid communication with the sample collecting chamber inlet so that the fluid communication between the second valve outlet and the sample collecting chamber inlet extends through the milk flow-through unit and
wherein the second milk flow-through unit outlet is in fluid communication with the milk line at a position downstream of the main valve,
wherein the milk flow-through unit is configured so that a milk stream that is supplied to the milk flow-through unit inlet of the milk flow-through unit splits into a first milk stream and a second milk stream that leave the milk flow-through through unit,. respectively,. via the first milk flow-through unit outlet and the second milk flow-through unit outlet, the flow of the first milk stream and the second milk stream having a predetermined ratio so that, in use, when the main valve is in the second state, a portion of the milk that flows through the second valve outlet is supplied to the sample collecting chamber by way of the first milk stream and a portion of the milk that flows through the second valve outlet is supplied to the milk line at a position downstream of the main valve by way of the second milk stream for return to the milk line.

5. The apparatus according to claim 4, wherein the milk flow-through unit comprises:
a chamber with a ceiling,
a first upstanding line section that comprises an open end at an upper side and of which an open underside is connected with the first milk flow-through unit outlet,
a second upstanding line section that comprises an open end at an upper side and of which an open underside is connected with the milk flow-through unit inlet, and
a bottom with an outflow opening that is connected with the second milk flow-through unit outlet,
wherein the milk flow-through unit is configured so that a milk stream that is supplied to the milk flow-through unit inlet is, inside the chamber, upwardly directed and via the second upstanding line section spouts against the ceiling to then leave the milk flow-through unit respectively via the open end at the upper side of the first upstanding line section, the rest of the first upstanding line section and the first milk flow-through unit outlet as the first milk stream and via the outflow opening and the second milk flow-through unit outlet as the second milk stream.

6. The apparatus according to claim 4, wherein the milk flow-through unit comprises a chamber provided with:
the milk flow-through unit inlet,
the first milk flow-through unit outlet, and
the second milk flow-through unit outlet;
wherein the milk flow-through unit inlet and the second milk flow-through unit outlet-464 are opposite to each other,
wherein the first milk flow-through unit outlet is in between the milk flow-through unit inlet and the second milk flow-through unit outlet,
wherein the milk flow-through unit is configured so that a milk stream which that is supplied to the milk flow-through unit inlet leaves the milk flow-through unit via the first milk flow-through unit outlet in the first milk stream and via the second milk flow-through unit outlet in the second milk stream.

7. The apparatus according to claim 6, wherein the milk flow-through unit comprises a channel formed by the chamber, that extends, from the milk flow-through unit inlet to the second milk flow-through unit outlet,
wherein the channel at the location of the first milk flow-through unit outlet is provided with a milk stream deflecting element, for deflecting, to the first milk flow-through unit outlet, a part of the milk stream that flows in the direction from the milk flow-through unit inlet.

8. The apparatus according to claim 7, wherein the milk stream deflecting element is approximately halfway along a length of the channel, viewed in a flow-through direction of the channel.

9. The apparatus according to claim 1, wherein the apparatus comprises a control unit for controlling the main valve from a first state to its a second state and vice versa, and controlling other valves of the apparatus.

10. The apparatus according to claim 1, wherein the second valve outlet is directly in fluid communication with the sample collecting chamber inlet,
wherein the apparatus includes a control unit for controlling the main valve from a first state to a second state and vice versa.

11. The apparatus according to claim 10, wherein the control unit controls the main valve with a pattern from the first state to the second state and vice versa, that the sample collecting chamber, in use, is filled with partial samples that in combination represent a picture of the total amount of milk that flows through the valve inlet of the main valve.

12. The apparatus according to claim 10, wherein the control unit controls the main valve with a pattern from the first state to the second state and vice versa, so that the sample collecting chamber, in use, is filled with partial samples of a same volume and with a spread in time in which the partial samples are taken,
wherein the frequency of taking samples is proportional to magnitude of flow of the milk stream through the valve inlet.

13. The apparatus according to claim 10, wherein the control unit controls the main valve with a pattern from the first state to the second state and vice versa, so that the sample collecting chamber, in use, is filled with partial samples where magnitude of a volume in each case is proportional to magnitude of flow of the milk stream through the valve inlet, while the partial samples are taken at a fixed frequency.

14. The apparatus according to claim 1, wherein the apparatus further comprises a sample valve provided with:
a valve inlet,
a first valve outlet, and
a second valve outlet,
wherein the second valve outlet of the main valve is in fluid communication with the valve inlet of the sample valve,
wherein the first valve outlet of the sample valve is in fluid communication with the collecting chamber inlet, and
wherein the second valve outlet of the sample valve is in fluid communication with the milk line downstream of the main valve,
wherein the sample valve is configured to selectively switch between a first state and a second state,
wherein in the first state the first valve inlet of the sample valve is in fluid communication with the first valve outlet of the sample valve so that the milk can flow through the sample valve to the sample collecting chamber, and
wherein in the second state the first valve inlet of the sample valve is in fluid communication with the second valve outlet of the sample valve for supplying milk that flows through the sample valve to the milk line downstream of the main valve.

15. The apparatus according to claim 14, wherein the control unit, when the main valve is in the second state, switches the sample valve with a pattern between the first state and the second state so that the sample collecting chamber, in use, is filled with partial samples that represent in combination a picture of a total amount of milk flowing through the valve inlet.

16. The apparatus according to claim 14, wherein the control unit, when the main valve is in the second state, switches the sample valve with a pattern between the first state and the second state so that the sample collecting chamber, in use, is filled with partial samples of a same volume and with a spread in the time in which the partial samples are taken,
wherein the frequency of taking samples is proportional to magnitude of-the flow of the milk stream through the valve inlet.

17. The apparatus according to claim 14, wherein the control unit, when the main valve is in the second state, switches the sample valve with a pattern between the first state and the second state so that the sample collecting chamber, in use, is filled with partial samples of volumes that are proportional to magnitude of flow of the milk stream through the valve inlet of the main valve, while the partial samples are taken at a fixed frequency.

18. The apparatus according to claim 1, wherein the apparatus further comprises a sample take-up chamber having a volume that is smaller than the sample collecting chamber, and
wherein the sample take-up chamber, for via a tenth line in which a second switchable open-close valve is included, can be filled from the sample collecting chamber.

19. The apparatus at least according to claims 2, wherein the apparatus further comprises a sample take-up chamber whose volume is smaller than the sample collecting chamber and wherein the sample take-up chamber can be filled from the sample collecting chamber,
wherein the second line and the tenth line are each connected with the outlet of the second switchable open-close valve.

20. The apparatus according to claim 18, wherein the tenth line is connected with the sample collecting chamber via a second sample collecting chamber outlet.

21. The apparatus according to claim 18, wherein the sample collecting chamber is provided with an air inlet opening for supplying air to the sample collecting chamber while milk flows from the sample collecting chamber into the sample take-up chamber.

22. The apparatus according to claim 18, wherein the sample take-up chamber is provided with a dispensing outlet configured for selectively dispensing or not dispensing the milk that is in the sample take-up chamber into an external receptacle.

23. The apparatus according to claim 1, wherein the main valve is further provided with a second valve inlet, wherein in a second state the second valve inlet is in fluid communication with the first valve outlet, wherein the at least one sample collecting chamber outlet is directly in fluid communication with the second valve inlet and hence with the milk line at a position downstream of the main valve while the main valve is in the second state so that in the second state the excess milk can be led back from the sample collecting chamber via the main valve to the milk line.

24. The apparatus according to claim 4, wherein the main valve is further provided with a second valve inlet, wherein in a second state the second valve inlet is in fluid communication with the first valve outlet, wherein the at least one sample collecting chamber outlet is directly in fluid communication with the second valve inlet and hence with the milk line at a position downstream of the main valve while the main valve is in the second state so that in the second state the excess milk can be led back from the sample collecting chamber via the main valve to the milk line, and
wherein the second milk flow-through unit outlet is directly in fluid communication with the second valve inlet for, in the second state of the main valve, supplying the second milk stream via the main valve to the milk line downstream of the main valve.

25. The apparatus according to claim 14, wherein the main valve is further provided with a second valve inlet, wherein in a second state the second valve inlet is in fluid communication with the first valve outlet, wherein the at least one sample collecting chamber outlet is directly in fluid communication with the second valve inlet and hence with the milk line at a position downstream of the main valve while the main valve is in the second state so that in the second state the excess milk can be led back from the sample collecting chamber via the main valve to the milk line, and
wherein the second valve outlet of the sample valve is directly in fluid communication with the second valve inlet for, in the second state of the main valve, supplying milk to the milk line downstream of the main valve.

26. The apparatus according to claim 1, wherein the sample collecting chamber is configured for mixing the milk in the sample collecting chamber.

27. The apparatus according to claim 1, wherein the sample collecting chamber is provided with an air supply opening configured to be opened and closed to, in a settable manner, supply or not supply air to the sample collecting chamber, wherein the air supply opening is at an underside of the sample collecting chamber.

28. The apparatus according to claim 1, wherein the flow of the first milk stream is within a range of 1-5%, in particular 2%, of the flow of the milk stream which that is supplied to the first milk inlet of the main valve.

29. The apparatus according to claim 11, wherein the flow that is formed by taking the partial samples is within a range of 1-5% of the flow of the milk stream that is supplied to the first milk inlet of the main valve.

30. The apparatus according to claim 1, wherein the apparatus is provided with an inlet that is in fluid communication with the first valve inlet of the main valve and an outlet that is in fluid communication with the first valve outlet,
wherein, for the operation of the apparatus, an existing milk line need only be cut open at one point to connect the apparatus with the milk line, so that milk in the apparatus and that does not belong to or is not going to belong to a sample can flow back to the milk line via the outlet.

31. The apparatus according to claim 1, wherein the apparatus is further provided with a fluid communication that is configured to be connected with a vacuum pump (9) for lowering the pressure in the sample collecting chamber.

32. A system comprising:
an apparatus for taking a sample of milk that flows through a milk line, wherein:
the apparatus comprises a main valve comprising a first valve inlet and a first valve outlet,
the apparatus is configured for including the main valve in a milk flow path of the milk line by placing the first valve inlet in fluid communication with a first open end of the milk line and placing the first valve outlet in fluid communication with a second open end of the milk line, the first open end being located upstream of the second open end in the milk flow path,
the apparatus further comprises a sample collecting chamber for receiving the sample of the milk, the sample collecting chamber comprising:
a sample collecting chamber inlet, and at least one sample collecting chamber outlet;
the main valve further comprises a second valve outlet in fluid communication with the sample collecting chamber inlet,
the main valve is configured to selectively switch between a first state and a second state, wherein in the first state the first valve inlet is in fluid communication with the first valve outlet so that the milk can flow through the milk line without a sample being taken, and wherein in the second state the first valve inlet is in fluid communication with the second valve outlet for supplying milk from the milk line to the sample collecting chamber, and
the apparatus is configured to selectively place or not place the sample collecting chamber outlet in fluid communication with the milk line at a position downstream of the main valve for leading back to the milk line excess milk of a milk sample that is in the sample collecting chamber; and
an external receptacle,
wherein the receptacle is provided with an identification code.
